(19) United States
(12) Patent Application Publication
(10) Pub. No.: US 2006/0182822 A1
(43) Pub. Date: Aug. 17, 2006

(54) Use of a soluble lucerne extract in order to slow weight gain in mammals

(76) Inventor: Didlier Coulmier, Chalons-En-Champagne (FR)

Correspondence Address: YOUNG & THOMPSON, 745 SOUTH 23RD STREET, 2ND FLOOR, ARLINGTON, VA, 22202, US

(21) Appl. No.: 10/557,020

(22) PCT Filed: May 14, 2004
(86) PCT No.: PCT/FR04/01203

§ 371 Date: Mar. 15, 2006

(30) Foreign Application Priority Data
May 16, 2003 (FR) 03/05924

(51) Int. Cl. A61K 36/48

(52) U.S. Cl. 424/757

(57) ABSTRACT

A method uses a soluble Lucerne extract obtained by pressing fresh Lucerne, containing at least 1 wt. % saponines in relation to the weight of the extract, for the production of a food preparation which can limit weight gain in mammals, partially in the case of a hyperlipemic diet, the preparation being adapted for addition to the food rations of the mammal in doses of the extract amounting to 0.8 g to 3 g per day and per kilogram of mammal body weight.

The present invention relates to the use of a soluble alfalfa extract in the field of human and animal nutrition, and in particular for mammals.

The way people eat in the western part of the world often results in various unbalances that are revealed by certain signs amongst which obesity and high blood cholesterol levels, inducing many diseases.

Synthetic materials developed by the pharmaceutical industry to fight against such metabolism dysfunctions have in many cases a plurality of side effects a certain number of which may be lethal.

The weight gain as the cholesterolemia of a subject partly depends on its eating habits and especially on the ingested lipid portion.

Blocking part of the lipids during the digestion process resulting in an intestine non-absorption makes it possible to reduce the ration energy supply as lipids, and to reduce cholesterol precursor penetration into the system.

It is known by the one skilled in the art that saponins, that are found especially in many plant species have cholesterol-lowering effects.

Indeed, according to the article entitled “Alfalfa saponins and their Implication in Animal Nutrition>>, J. Agric. Food Chem. 1998, 46, 131-140, it is known that administrating saponins to animals such as chicks or adult cocks makes it possible to lower the blood plasma cholesterol level. Similarly, cholesterol ingestion mediated hypercholesterolemia can be prevented in rabbits by incorporating saponins into the diet. Nevertheless, there is no reference in this article to any effect of saponins on weight gain. Moreover, there is nothing about any saponin daily dosage needed for obtaining a significant effect on the blood cholesterol level.

Moreover, the French patent application FR 2,794,616 describes especially an alfalfa derived plant product, having a certain saponin or sapogenin concentration, and that is free from the unfortunately toxic carnavanin that comes with. The French patent application FR 2,794,616 also describes how to use this plant product in a method for preparing a nutritional supplement with cholesterol-lowering properties so as to the cholesterol that is present or coming into the intestine. In this patent application, it is stated that supplying a human subject once a day with this plant product comprising from 1.5 to 40% by weight saponins or sapogenins, that is to say from 50 to 1 000 mg per day, and in particular from 200 to 400 mg per day, enables to maintain the plasma cholesterol at a normal level, even if the subject ingesting it has a diet containing food products that are well known for their high cholesterol content. Nevertheless, there is nothing in this application about any effect of this product on weight gain limitation that would be induced by such a diet. Moreover, the indicated dosage does not account for the body weight of the subject.

Now the applicant has discovered that administrating to rats a soluble alfalfa extract comprising at least 1% saponin, that is to say from 0.8 to 3 g per day and per kg of leave animal weight in the context of a lipid-raising diet, could restrain the weight gain. The applicant also discovered that this could lower blood total cholesterol and triglyceride levels, while maintaining the VLDL cholesterol level at constant values despite the lipid-raising diet.

As used herein, VLDL cholesterol level (also called “bad cholesterol”) means the cholesterol level comprised in the very low density lipoproteins (VLDL).

So, it is therefore an object of the present invention to provide the use of a soluble alfalfa extract produced by pressing fresh alfalfa, and comprising at least 1% by weight saponins as compared to the extract weight, to restrict the weight gain in mammals, especially in the context of a lipid-raising diet, consisting in adding to the mammal's food ration from 0.8 to 3 g, preferably from 0.9 g to 2.73 g, and more preferably 0.91 g per day and per kg of mammal's live weight of said extract.

Preferably, the soluble alfalfa extract used according to the present invention is in the form of a powder, the moisture content of which ranges from 3 to 10% by weight, and comprises by weight as compared to the weight of dry powder:
from 15% to 25% raw proteins,
from 10% to 30% inorganic materials,
from 0% to 2% raw cellulose,
from 10% to 25% sugars, and
from 5% to 20% organic acids.

Inorganic materials are traditionally based on potassium, calcium, magnesium, phosphorus, zinc and iron.

Sugars comprised in the soluble alfalfa extract used according to the present invention generally are mainly represented by glucose and fructose, and to a lower extent by galactose, stachyose and verbascose.

The soluble alfalfa extract advantageously comprises, as expressed by weight compared to the weight of dry powder, from 5 to 12% malic acid.

The soluble alfalfa extract used according to the present invention may be obtained by means of the method described in the French patent FR 2,294,647. It generally corresponds to an alfalfa juice prepared by pressing, the precipitable proteins of which have been removed, and that has been then concentrated. The source alfalfa preferably does not undergo any prewithering, and so can preserve the major part of metabolic compounds present in the plant (vitamins, growth factors, intermediate metabolites, for example) as it is harvested.

In particular, the soluble alfalfa extract may be obtained by concentrating, then drying a serum prepared from fresh alfalfa according to a method comprising following steps consisting in:

(a′) fresh alfalfa cold pressing, whereupon a green juice and a cake are produced;

(b′) treating the green juice by alkalinizing it to a pH around 8.5, for example with ammoniac;

(c′) flocculating green juice proteins by means of a heat treatment, thus providing a protein precipitate and a soluble extract (also called serum);

(d′) centrifugating the soluble extract-precipitated protein mixture until a soluble alfalfa extract (or serum) is obtained;

(e′) spray drying the soluble alfalfa extract, whereupon a dry extract is obtained as a ocher-colored powder.

The soluble alfalfa extract is preferably incorporated into a (liquid or solid) food preparation before being administrated via the oral route, thus supplementing the food ration.

Indeed, the soluble alfalfa extract such as previously defined enables a preparation to be made for restricting the weight gain in mammals, said preparation being suitable for said extract to be added to the mammal's food ration in an amount ranging from 0.8 g to 3 g per day and per kg of mammal's live weight.

Using an alfalfa extract according to the present invention such as previously defined makes it further possible, simultaneously with the weight gain restriction, to lower cholesterol and triglyceride blood total levels, and to maintain blood VLDL cholesterol at a constant level, even in the context of a lipid-raising diet.

The following example without being limitative illustrates the present invention in more details.

### EXAMPLE

Evaluating the effects of an alfalfa extract administrated via the oral route, on Wistar adult male rat weight gain and lipemia.

In this example, the effects of consuming 30 days long an alfalfa extract (or alfalfa serum) comprising 1.1% by weight saponins as compared to the extract weight, on Wistar adult male rat weight gain and lipemia were assessed.

Three groups each containing 12 adult male rats were fed for 45 days a hyperlipidic diet so as to induce a relative hyperlipemia (triglycerides and cholesterol). Two of these groups received the alfalfa serum from the 15^{th} to the 45^{th} day. The third rat group which was not treated with the alfalfa serum was used as a control.

Blood samples were collected during the lipemia induction on days 0, 7 and 15 and during the treatment with the alfalfa serum on days 30 and 45, to determine the lipidic state of the animals.

1. Subjects

Thirty-six Wistar adult male rats/AF EOPS (breeding center Charles River/Iffa-Credo, 69-St-Germain sur l'Arbresle, France) aged 16 weeks.

The rats were marked and each group of two individuals was distributed in polycarbonate F type cages (43×28×15 cm, U.A.R., 91—Epinay-sur-orge, France). The animals were stabulated in an acclimatized animal room, at a temperature ranging from 22 to 24° C. and underwent a reverse 12 hours light-dark cycle (light between 8 p.m. and 8 a.m.).

Once familiarized for a week with the experimental conditions and after a 15 day-long lipid-raising treatment, rats were distributed in 3 treatment groups in coherence with their lipidic status (cholesterol and triglycerides):

a control group (comprising 12 rats), and

two Alfalfa serum groups (each also comprising 12 rats).

The rats belonging to the three groups were all handled in the same way and in similar conditions. To prevent any possible interference between the various products, both rats in the same cage received the same product.

2. Test product Soluble alfalfa extract provided as a dry powder by ALFALIS.

The product characteristics are listed hereafter in table 1.

TABLE 1

| | |
|---|---|
| Product | Alfalfa serum |
| Source | ALFALIS |
| | France |
| Batch number | 05877 |
| Expiration date | Unknown |
| Storage | At ambient temperature, |
| | kept away from light, |
| | moisture and mold |

3. Experience Procedure

Drinking water and hyperlipidic diet specifically prepared to raise the lipemia in the animals were proposed ad libitum to the animals.

This diet comprised standard feed (feed M20, Dietex, France) supplemented with lipids in the form of refined pork fat (lard 21.4%), with cholesterol (1%, Acros Organics, Noisy-le-Grand, France), as well as in saccharose (3.8%). The prepared diet was reconstituted as pellets by adding 20% water.

Rats of both Alfalfa serum groups all daily received from the 15th to the 45^{th} day, 0.91 g and 2.73 g/kg of live weight of Alfalfa serum extract, comprising 10 and 30 mg saponins/kg.

Control rats daily treatment comprised a solution containing 0.54 g/kg saccharose corresponding to the carbohydrate portion contained in the alfalfa serum extract in the most concentrated dose.

On days 0, 7, 15, 30 and 45, the rats belonging to the three groups remained in a fasted state 12 hours long before collecting blood samples.

The alfalfa extract administration protocol is detailed hereafter in table 2.

TABLE 2

| | Number of | | | Administration |
|---|---|---|---|---|
| | rats | Dose | Administration days | days for alfalfa |
| Groups | per group | (g/kg) | for hyperlipidic diet | extract |
| Vehicle | 12 | — | From D1 to D45 | From D15 to D45 |
| Alfalfa 1 | 12 | 0.91 | | |
| Alfalfa 2 | 12 | 2.73 | | |

4—Product Administration

The “Alfalfa serum” extract was suspended in an aqueous solution and administrated via the oral route once a day from the 15th to the 45^{th} day.

5—Analysis Variables

To apply the alfalfa extract effect on the WISTAR male rat lipemia, following biological data were assayed in the serum: the total cholesterol level, the triglyceride level and the VLDL cholesterol level.

The animals were weighted and values recorded every three or four days so as to adjust the treatment doses.

The rat food intake was evaluated once a week.

6. Results

Weight Evolution

The weight evolution (in g) for the 3 groups of rats is expressed in mean weight (in g) from day 1 to day 43, on FIG. 1 and results are listed in table 3.

FIG. 1 and table 3 demonstrate the following:
for the first 15 days of treatment with the lipid-raising diet, the rat weight for all the three groups changed in a similar way;
from day 18, and until the end of treatment, the weight of the Alfalfa 2 group rats remained substantially lower than the one of the control group rats;
from day 25 and until the end of treatment, the weight of the Alfalfa 1 group rats remained substantially lower than the one of the control group rats.

The treatment with the alfalfa extract enabled to restrict the weight gain following a lipid-raising diet. The weight gain restriction effect was stronger when the alfalfa extract was administrated in an amount corresponding to 2.73 g/kg (Alfalfa 2), than to 0.91 g/kg (Alfalfa 1).

TABLE 3
Weight evolution (g) (mean ± SEM)

| | Vehicle | Alfalfa 1 | Alfalfa 2 |
|---|---|---|---|
| Days | (n = 12) | (n = 12) | (n = 9) |
| D1 | 357.00 ± 2.37 | 356.17 ± 1.97 | 357.44 ± 3.38 |
| D5 | 376.33 ± 3.07 | 376.67 ± 3.04 | 376.44 ± 4.48 |
| D8 | 388.83 ± 4.75 | 390.92 ± 4.14 | 387.44 ± 5.08 |
| D11 | 410.42 ± 5.45 | 414.08 ± 5.15 | 409.11 ± 5.49 |
| D14 | 426.25 ± 6.14 | 428.25 ± 5.38 | 421.00 ± 6.64 |
| D18 | 439.33 ± 7.07 | 429.33 ± 6.71 | 413.44 ± 9.22 |
| D22 | 456.25 ± 7.05 | 440.25 ± 7.40 | 429.00 ± 10.12 |
| D25 | 463.83 ± 6.97 | 442.50 ± 6.83 | 428.11 ± 9.18 |
| D28 | 473.00 ± 7.06 | 448.75 ± 7.68 | 435.00 ± 8.91 |
| D32 | 485.28 ± 7.28 | 458.08 ± 7.37 | 442.67 ± 10.24 |
| D35 | 491.33 ± 7.71 | 462.92 ± 7.54 | 447.56 ± 10.81 |
| D39 | 504.17 ± 7.77 | 475.75 ± 8.53 | 455.67 ± 11.36 |
| D43 | 510.00 ± 8.26 | 481.67 ± 10.00 | 457.00 ± 11.78 |

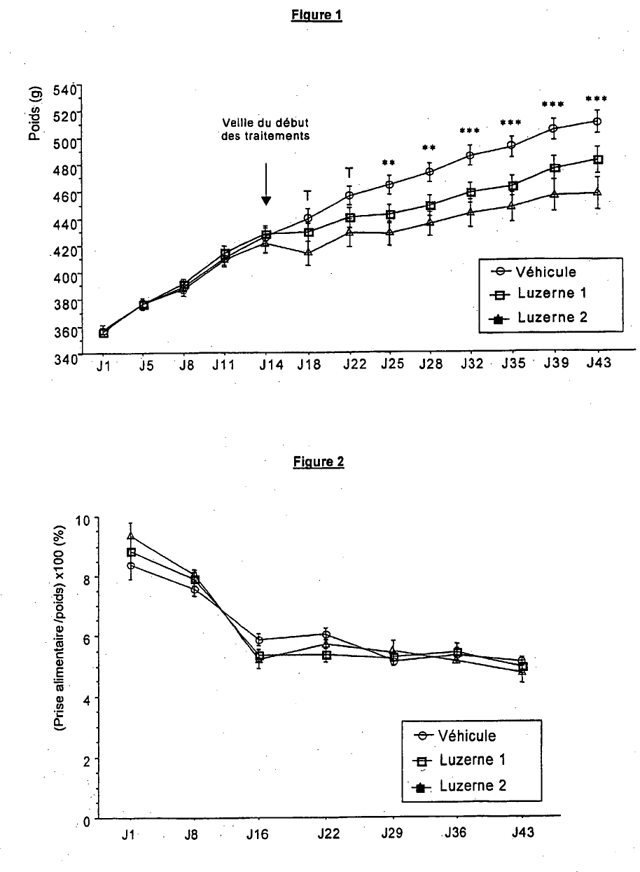

Food Intake Evolution The food intake evolution from day 1 to day 43 for the 3 groups of rats is illustrated as the mean food intake as compared to the body weight (in %) on FIG. 2 and corresponds to the results listed in table 4.

FIG. 2 and table 4 demonstrate that on days 1, 8, 16, 22, 29, 36 and 43, there was no significant difference in food intake amongst the 3 groups of rats.

The rats that were treated with the alfalfa extract gained substantially less weight than the control rats did, while the rats belonging to the 3 groups had eaten in the same amount as compared to their respective weights.

TABLE 4
Food intake evolution according to
body weight (%) (mean ± SEM)

| | Vehicle | Alfalfa 1 | Alfalfa 2 |
|---|---|---|---|
| Days | (n = 12) | (n = 12) | (n = 9) |
| D1 | 8.41 ± 0.49 | 8.86 ± 0.41 | 9.37 ± 0.43 |
| D8 | 7.58 ± 0.25 | 7.92 ± 0.24 | 8.07 ± 0.18 |
| D16 | 5.87 ± 0.21 | 5.36 ± 0.21 | 5.22 ± 0.31 |
| D22 | 6.04 ± 0.18 | 5.34 ± 0.21 | 5.71 ± 0.14 |
| D29 | 5.12 ± 0.15 | 5.26 ± 0.15 | 5.48 ± 0.32 |
| D36 | 5.34 ± 0.23 | 5.42 ± 0.27 | 5.13 ± 0.09 |
| D43 | 5.10 ± 0.15 | 4.92 ± 0.21 | 4.72 ± 0.34 |

Total Cholesterol Level Evolution

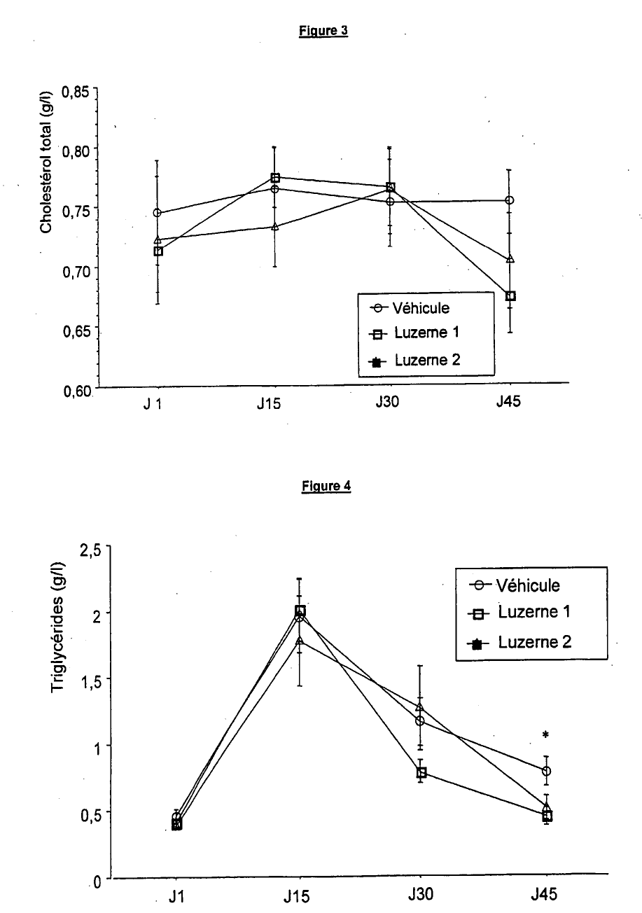

The total cholesterol level evolution for the rats belonging to the 3 groups, from day 1 to day 43, is illustrated as the blood mean total cholesterol level (in g/l) on FIG. 3 and corresponds to the results listed in table 5.

These results reveal the following:
for the lipid-raising diet 45 days of treatment the total cholesterol level remained constant for the rats belonging to the Vehicle and Alfalfa 2 groups;
for the rats of the Alfalfa 1 group, the total cholesterol level lowered substantially from D30 to D45;

The treatment with the alfalfa extract for 30 days in an amount corresponding to 0.91 g/kg lowered the total cholesterol level.

TABLE 5
Total cholesterol level evolution (g/l) (mean ± SEM)

| | Vehicle | Alfalfa 1 | Alfalfa 2 |
|---|---|---|---|
| Days | (n = 12) | (n = 12) | (n = 9) |
| D1 | 0.75 ± 0.04 | 0.73 ± 0.04 | 0.72 ± 0.54 |
| D15 | 0.76 ± 0.03 | 0.77 ± 0.03 | 0.73 ± 0.03 |
| D30 | 0.75 ± 0.04 | 0.77 ± 0.03 | 0.76 ± 0.04 |
| D45 | 0.75 ± 0.03 | 0.67 ± 0.03 | 0.70 ± 0.04 |

Triglyceride Level Evolution

The triglyceride level evolution from day 1 to day 43 is illustrated as the blood mean level in g/l and illustrated on FIG. 4 and corresponds to the results listed in table 6.

These results reveal the following:
for the first 15 days of treatment with the lipid-raising diet, the triglyceride level increased substantially for the rats belonging to the 3 treatment groups;
from D15 to D30, it lowered substantially for the rats of the Vehicle and Alfalfa 1 groups;
from D30 to D45, it lowered substantially for the rats of the Alfalfa 1 and Alfalfa 2 groups;
the last day of the treatments (D45), the triglyceride level was lower for the rats of the Alfalfa 1 and Alfalfa 2 groups than for the rats of the control group.

The treatment with the alfalfa extract for 30 days enabled to substantially lower the triglyceride level when administrated in an amount corresponding to 0.91 g/kg and tended to lower when administrated in an amount corresponding to 2.73 g/kg.

TABLE 6
Triglyceride level evolution (g/l) (mean ± SEM)

| | Vehicle | Alfalfa 1 | Alfalfa 2 |
|---|---|---|---|
| Days | (n = 12) | (n = 12) | (n = 9) |
| D1 | 0.46 ± 0.05 | 0.41 ± 0.03 | 0.38 ± 0.02 |
| D15 | 1.96 ± 0.28 | 2.01 ± 0.24 | 1.77 ± 0.35 |
| D30 | 1.15 ± 0.18 | 0.78 ± 0.09 | 1.26 ± 0.32 |
| D45 | 0.78 ± 0.11 | 0.44 ± 0.06 | 0.50 ± 0.09 |

VLDL Cholesterol Level Evolution

The VLDL cholesterol level evolution, from day 1 to day 43, for the 3 groups of rats is illustrated as the blood mean level illustrated on FIG. 5, and corresponds to the results listed in table 7.

The results reveal that:
for the first 15 days of the treatment with the lipid-raising diet, the VLDL cholesterol level remained constant for the rats of the Vehicle and Alfalfa 1 groups, but increased substantially for the rats of the Alfalfa 2 group. This increase resulted from a marked individual variability amongst this group independently from the alfalfa administration which only started on day 15;
from D15 to D45, the VLDL cholesterol level tended to increase for the control rats despite a tendency to decrease between D30 and D45;
on day 45, the VLDL cholesterol level for the control rats was substantially higher than that of the rats belonging to the Alfalfa 1 group and tended to be higher than that of the rats belonging to the Alfalfa 2 group;
from D15 to D45, the VLDL cholesterol level tended to decrease for the rats belonging to the Alfalfa 2 group, then lowered substantially from D30 to D45 and became basically normal again;
during the treatment time, the VLDL cholesterol level remained constant for the rats belonging to the Alfalfa 1 group.

The treatment with the alfalfa extract for 30 days enabled to maintain the VLDL cholesterol level at constant values despite the lipid-raising diet.

Treating the rats with alfalfa extract seemed to be more efficient with a dose corresponding to 0.91 g/kg/day as compared with a dose corresponding to 2.73 g/kg/day.

TABLE 7
VLDL cholesterol level evolution (g/l) (mean ± SEM)

| | Vehicle | Alfalfa 1 | Alfalfa 2 |
|---|---|---|---|
| Days | (n = 12) | (n = 12) | (n = 9) |
| D1 | 0.09 ± 0.01 | 0.08 ± 0.01 | 0.08 ± 0.003 |
| D15 | 0.10 ± 0.04 | 0.11 ± 0.04 | 0.22 ± 0.05 |
| D30 | 0.23 ± 0.04 | 0.16 ± 0.02 | 0.18 ± 0.05 |
| D45 | 0.16 ± 0.02 | 0.09 ± 0.01 | 0.10 ± 0.02 |

The effects of the alfalfa extract intake for 30 days on the weight gain and the lipemia for the Wistar male adult rats hence were as follows:
weight gain restriction following a lipid-raising diet;
with a dose corresponding to 0.91 g/kg, total cholesterol level lowering;
significant lowering of the triglyceride level when the alfalfa extract was administrated in an amount corresponding to 0.91 g/kg and tendency to lower when administrated in an amount corresponding to 2.73 g/kg;
VLDL cholesterol level remaining constant despite the lipid-raising diet; the alfalfa extract seemed to be more efficient at a dose corresponding to 0.91 g/kg/day than to 2.73 g/kg/day.

1. A method for restricting weight gain in mammals, comprising adding an extract to the mammal's food ration in an amount ranging from 0.8 g to 3 g per day and per kg of mammal's live weight, wherein the extract is a soluble alfalfa extract obtained by pressing fresh alfalfa and comprising at least 1% by weight saponins as compared to the extract weight.

2. The method according to claim 1, characterized in that said food preparation is suitable for said extract to be added to the food ration in an amount ranging from 0.9 g to 2.73 g, per day and per kg of mammal's live weight.

3. The method according to claim 1, characterized in that the soluble alfalfa extract comes as a powder, the moisture content of which ranges from 3 to 10% by weight, said extract comprising, as expressed by weight compared to the weight of dry powder:
from 15% to 25% raw proteins,
from 10% to 30% inorganic materials,
from 0% to 2% raw cellulose,
from 10% to 25% sugars, and
from 5% to 20% organic acids.

4. The method according to claim 3, characterized in that the soluble alfalfa extract comprises, as expressed by weight compared to the weight of dry powder, from 5 to 12% malic acid.

5. The method according to claim 1, characterized in that the soluble alfalfa extract is obtained by concentrating and drying a serum derived from fresh alfalfa according to a method comprising following steps consisting in:
(a′) fresh alfalfa cold pressing, whereupon a green juice and a cake are produced;
(b′) treating the green juice by alkalinizing it to a pH around 8.5, for example with ammoniac;
(c′) flocculating green juice proteins by means of a heat treatment, thus providing a protein precipitate and a soluble alfalfa extract;
(d′) centrifugating the soluble extract—precipitated proteins mixture until the soluble alfalfa extract is obtained;
(e′) spray drying the serum, thus providing a dry extract as a ocher-colored powder.

6. The method according to claim 1, characterized in that said extract is incorporated into the food preparation before being administrated via the oral route, as a supplement to the food ration.

7. The method according to claim 1, characterized in that, simultaneously to the weight gain restriction, said extract also enables to lower the blood total cholesterol level.

8. The method according to claim 1, characterized in that, simultaneously to the weight gain restriction, said extract also enables to maintain the blood VLDL cholesterol at a constant level.

9. The method according to claim 1, characterized in that, simultaneously to the weight gain restriction, said extract also enables to lower the blood triglyceride level.

10. The method according to claim 2, characterized in that the soluble alfalfa extract comes as a powder, the moisture content of which ranges from 3 to 10% by weight, said extract comprising, as expressed by weight compared to the weight of dry powder:
from 15% to 25% raw proteins,
from 10% to 30% inorganic materials,
from 0% to 2% raw cellulose,
from 10% to 25% sugars, and
from 5% to 20% organic acids.

11. The method according to claim 2, characterized in that the soluble alfalfa extract is obtained by concentrating and drying a serum derived from fresh alfalfa according to a method comprising following steps consisting in:
(a′) fresh alfalfa cold pressing, whereupon a green juice and a cake are produced;
(b′) treating the green juice by alkalinizing it to a pH around 8.5, for example with ammoniac;
(c′) flocculating green juice proteins by means of a heat treatment, thus providing a protein precipitate and a soluble alfalfa extract;
(d′) centrifugating the soluble extract—precipitated proteins mixture until the soluble alfalfa extract is obtained;
(e′) spray drying the serum, thus providing a dry extract as a ocher-colored powder.

12. The method according to claim 3, characterized in that the soluble alfalfa extract is obtained by concentrating and drying a serum derived from fresh alfalfa according to a method comprising following steps consisting in:
(a′) fresh alfalfa cold pressing, whereupon a green juice and a cake are produced;
(b′) treating the green juice by alkalinizing it to a pH around 8.5, for example with ammoniac;
(c′) flocculating green juice proteins by means of a heat treatment, thus providing a protein precipitate and a soluble alfalfa extract;
(d′) centrifugating the soluble extract—precipitated proteins mixture until the soluble alfalfa extract is obtained;
(e′) spray drying the serum, thus providing a dry extract as a ocher-colored powder.

13. The method according to claim 4, characterized in that the soluble alfalfa extract is obtained by concentrating and drying a serum derived from fresh alfalfa according to a method comprising following steps consisting in:
(a′) fresh alfalfa cold pressing, whereupon a green juice and a cake are produced;
(b′) treating the green juice by alkalinizing it to a pH around 8.5, for example with ammoniac;
(c′) flocculating green juice proteins by means of a heat treatment, thus providing a protein precipitate and a soluble alfalfa extract;
(d′) centrifugating the soluble extract—precipitated proteins mixture until the soluble alfalfa extract is obtained;
(e′) spray drying the serum, thus providing a dry extract as a ocher-colored powder.

14. The method according to claim 2, characterized in that said extract is incorporated into the food preparation before being administrated via the oral route, as a supplement to the food ration.

15. The method according to claim 3, characterized in that said extract is incorporated into the food preparation before being administrated via the oral route, as a supplement to the food ration.

16. The method according to claim 4, characterized in that said extract is incorporated into the food preparation before being administrated via the oral route, as a supplement to the food ration.

17. The method according to claim 5, characterized in that said extract is incorporated into the food preparation before being administrated via the oral route, as a supplement to the food ration.

18. The method according to claim 2, characterized in that, simultaneously to the weight gain restriction, said extract also enables to lower the blood total cholesterol level.

19. The method according to claim 3, characterized in that, simultaneously to the weight gain restriction, said extract also enables to lower the blood total cholesterol level.

20. The method of claim 1, wherein the mammal is being fed a lipid-raising diet.
